(19) United States
(12) Patent Application Publication
(10) Pub. No.: US 2025/0299805 A1
(43) Pub. Date: Sep. 25, 2025

(54) INFORMATION PROCESSING APPARATUS, CARE NEEDS ASSESSMENT SUPPORT SYSTEM, AND RECORDING MEDIUM

(71) Applicant: NCE Corporation, Tokyo (JP)

(72) Inventor: Asako FUJII, Tokyo (JP)

(73) Assignee: NCE Corporation, Tokyo (JP)

(21) Appl. No.: 19/047,804

(22) Filed: Feb. 7, 2025

(30) Foreign Application Priority Data
Mar. 19, 2024 (JP) 2024-043910

(51) Int. Cl. G16H 40/20; G16H 10/20

(52) U.S. Cl. CPC G16H 40/20; G16H 10/20

(57) ABSTRACT

Determination of a degree of nursing care required is supported. An information processing apparatus includes: a query generating section which generates a query that instructs generation of a question sentence which asks about a matter serving as a basis for determining a degree of nursing care required; a generation control section which inputs, into a generative model that has been trained by machine learning to generate a question sentence in accordance with an input query, the query which is generated by the query generating section, and which causes the generative model to generate the question sentence; a presentation section which presents, to a target person, the question sentence that has been generated by the generative model; and a recording section that records an answer of the target person to the question sentence. This information processing apparatus even makes it possible to support decision making by a user.

This Nonprovisional application claims priority under 35 U.S.C. § 119 on Patent Application No. 2024-043910 filed in Japan on Mar. 19, 2024, the entire contents of which are hereby incorporated by reference.

### TECHNICAL FIELD

The present disclosure relates to an information processing apparatus, a care needs assessment support system, and a recording medium.

### BACKGROUND ART

A nursing care insurance system provides a care service in accordance with a nursing care level to an insured person who is in a state in need of support which state requires support for daily life. A nursing care level of an insured person is determined by secondary determination by an assessment committee member in a care needs assessment committee after primary determination by a computer on the basis of a result of a visiting survey by an assessment survey staff member and a primary care physician's written opinion.

Assessment of a nursing care level requires much manpower and time, that is, human and temporal cost for assessment of a nursing care level is high, and a technique for minimizing such cost is required. Examples of such a technique include a behavior analysis apparatus disclosed in Patent Literature 1 below. This behavior analysis apparatus analyzes a moving image of a target person whose nursing care level is to be assessed, and automatically determines the nursing care level on the basis of a result of analysis of the moving image.

### CITATION LIST

### Patent Literature

### Patent Literature 1

Japanese Patent Application Publication Tokukai No. 2022-25935

### SUMMARY OF INVENTION

### Technical Problem

However, a method for determining a nursing care level by the behavior analysis apparatus disclosed in Patent Literature 1 is not a publicly recognized determination method. Thus, it is considered impossible under the current system to receive provision of a care service on the basis of a nursing care level determined by the above determination method. Further, a case where a specific index, i.e., a nursing care level is determined and any other case where a degree of nursing care required is determined share the foregoing problem of cost.

An example object of the present disclosure is to provide a technique for supporting determination of a degree of nursing care required.

### Solution to Problem

An information processing apparatus in accordance with an example aspect of the present disclosure includes at least one processor, the at least one processor carrying out: a query generating process for generating a query that instructs generation of at least one question sentence which asks about a matter serving as a basis for determining a degree of nursing care required; a generation control process for inputting, into a generative model that has been trained by machine learning to generate a question sentence in accordance with an input query, the query which has been generated in the query generating process, and causing the generative model to generate the at least one question sentence; a presentation process for presenting, to a target person, the at least one question sentence that has been generated by the generative model; and a recording process for recording an answer of the target person to the at least one question sentence.

A care needs assessment support method in accordance with an example aspect of the present disclosure includes: a query generating process for generating a query that instructs generation of at least one question sentence which asks about a matter serving as a basis for determining a degree of nursing care required; a generation control process for inputting, into a generative model that has been trained by machine learning to generate a question sentence in accordance with an input query, the query which has been generated in the query generating process, and causing the generative model to generate the at least one question sentence; a presentation process for presenting, to a target person, the at least one question sentence that has been generated by the generative model; and a recording process for recording an answer of the target person to the at least one question sentence, the query generating process, the generation control process, the presentation process, and the recording process each being carried out by at least one processor.

A recording medium in accordance with an example aspect of the present disclosure is a non-transitory computer-readable recording medium recording therein a care needs assessment support program for causing a computer to function as: a query generating means for generating a query that instructs generation of at least one question sentence which asks about a matter serving as a basis for determining a degree of nursing care required; a generation control means for inputting, into a generative model that has been trained by machine learning to generate a question sentence in accordance with an input query, the query which is generated by the query generating means, and causing the generative model to generate the at least one question sentence; a presentation means for presenting, to a target person, the at least one question sentence that has been generated by the generative model; and a recording means for recording an answer of the target person to the at least one question sentence.

### Advantageous Effects of Invention

An example aspect of the present disclosure provides an example advantage of making it possible to support determination of a degree of nursing care required.

### BRIEF DESCRIPTION OF DRAWINGS

FIG. 1 is a block diagram illustrating a configuration of an information processing apparatus in accordance with the present disclosure.

FIG. 2 is a flowchart illustrating a flow of a care needs assessment support method in accordance with the present disclosure.

FIG. 3 is a view describing a configuration of a care needs assessment support system in accordance with the present disclosure.

FIG. 4 is a view describing support for a visiting survey by the care needs assessment support system in accordance with the present disclosure.

FIG. 5 is a block diagram illustrating a configuration of another information processing apparatus in accordance with the present disclosure.

FIG. 6 is a flowchart illustrating a flow of a process carried out by the information processing apparatus in accordance with the present disclosure.

FIG. 7 is a flowchart of a process in which the information processing apparatus in accordance with the present disclosure generates a survey report.

FIG. 8 is a flowchart of a process carried out by the information processing apparatus in accordance with the present disclosure in a visiting survey or a care needs assessment committee.

FIG. 9 is a block diagram illustrating a configuration of a computer that functions as the information processing apparatus in accordance with the present disclosure.

### EXAMPLE EMBODIMENTS

The following description will discuss example embodiments of the present invention. Note, however, that the present invention is not limited to the example embodiments described below, but can be altered in various ways by a skilled person in the art within the scope of the claims. For example, the present invention can also encompass, in its scope, any example embodiment derived by appropriately combining techniques (some or all of products or processes) employed in the example embodiments described below. Further, the present invention can also encompass, in its scope, any example embodiment derived by appropriately omitting some of the techniques employed in the example embodiments described below. Furthermore, effects mentioned in the example embodiments described below are example effects expected in the example embodiments described below, and are not intended to define an extension of the present invention. That is, the present invention can also encompass, in its scope, any example embodiment that does not bring about any of the effects mentioned in the example embodiments described below.

### First Example Embodiment

The following description will discuss a first example embodiment, which is an example embodiment of the present invention, in detail with reference to the drawings. The present example embodiment is a basic form of example embodiments described later. Note that the scope of application of techniques which are employed in the present example embodiment is not limited to the present example embodiment. That is, techniques which are employed in the present example embodiment can be employed also in the other example embodiments included in the present disclosure, provided that no particular technical problem occurs. Moreover, techniques which are indicated in the drawings referred to for describing the present example embodiment can be employed also in the other example embodiments included in the present disclosure, provided that no particular technical problem occurs.

### (Configuration of Information Processing Apparatus 1)

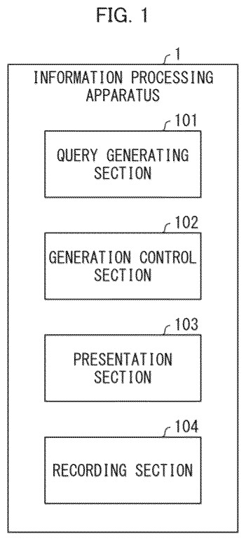

A configuration of an information processing apparatus 1 in accordance with the present example embodiment will be described with reference to FIG. 1. FIG. 1 is a block diagram illustrating the configuration of the information processing apparatus 1. The information processing apparatus 1 includes a query generating section 101, a generation control section 102, a presentation section 103, and a recording section 104 as illustrated in FIG. 1.

The query generating section 101 generates a query that instructs generation of a question sentence which asks about a matter serving as a basis for determining a degree of nursing care required. Matters serving as a basis for determining a degree of nursing care required may be publicly defined. For example, in a case where the information processing apparatus 1 is used to assess a nursing care level, the above matters are a physical function, a vital function, a cognitive function, and the like of a target person. Note that a method for generating the query need only be determined in advance. The information processing apparatus 1 can support not only determination of a specific index, i.e., a nursing care level, but also support any case where a degree of nursing care required is determined. For example, the information processing apparatus 1 can also support determination of another index indicating a degree of nursing care required, and can also support, as a precondition for determining a product or service in accordance with a degree of nursing care required, determination of the degree of nursing care required.

The generation control section 102 inputs, into a generative model, the query which is generated by the query generating section 101, and causes the generative model to generate a question sentence. The generative model need only have been trained by machine learning to generate the question sentence in accordance with an input query. For example, in a case where the query generating section 101 generates a query described in natural language, a language model that has learned, by machine learning, an arrangement of components (such as words) of a sentence described in natural language and an arrangement of sentences in text may be used as the generative model. Note that the generative model may be a model that is specialized in generating a question sentence which asks about a matter serving as a basis for determining a degree of nursing care required, or may be a general-purpose model that can also generate a sentence other than the question sentence.

The presentation section 103 presents, to a target person, the question sentence that has been generated by the generative model. A manner in which the question sentence is presented particularly limited. For example, the presenting section 103 may present the question sentence by display output through a display apparatus, may present the question sentence by audio output through an audio output apparatus, or may present the question sentence by print output through a printing apparatus. An output apparatus for use in presentation (e.g., the display apparatus, the audio output apparatus, or the printing apparatus) may be included in the information processing apparatus 1 or may be an apparatus external to the information processing apparatus 1.

Note that the target person to whom the question sentence is to be presented may be an assessment target person whose nursing care level is to be assessed, or may be a person who is different from the assessment target person and who understands an ordinary physical and mental state of the assessment target person whose nursing care level is to be assessed (e.g., a person living with the assessment target person, etc.).

The recording section 104 records an answer of the target person to the question sentence presented by the presentation section 103. The answer may be recorded in any storage apparatus. For example, the answer may be recorded in a storage apparatus included in the information processing apparatus 1, or may be recorded in a storage apparatus external to the information processing apparatus 1. Further, the answer may be input as text data or may be input as audio data. The recording section 104 may record, as it is, the answer input as audio data, or may convert the answer into text data and record the text data.

As described above, a configuration is employed such that the information processing apparatus 1 in accordance with the present example embodiment includes: the query generating section 101 which generates a query that instructs generation of at least one question sentence which asks about a matter serving as a basis for determining a degree of nursing care required; the generation control section 102 which inputs, into a generative model that has been trained by machine learning to generate a question sentence in accordance with an input query, the query which is generated by the query generating section 101, and which causes the generative model to generate the at least one question sentence; the presentation section 103 which presents, to a target person, the at least one question sentence that has been generated by the generative model; and the recording section 104 that records an answer of the target person to the at least one question sentence.

According to the above configuration, information serving as a basis for determining a degree of nursing care required can be collected and recorded without assistance from, for example, a survey staff member. Thus, the information processing apparatus 1 brings about an effect of making it possible to support determination of a degree of nursing care required. Further, in a case where the information processing apparatus 1 is used to assess a nursing care level, while using the above recorded information, it is possible to assess the nursing care level in accordance with a public framework (e.g., primary determination by a computer and secondary determination by an assessment committee member). Moreover, the information processing apparatus 1 also makes it possible to support decision making by a user of the information processing apparatus 1, such as a survey staff member or an assessment committee member.

### (Care Needs Assessment Support Program)

The foregoing functions of the information processing apparatus 1 can also be realized by a program. A care needs assessment support program in accordance with the present example embodiment causes a computer to function as: a query generating means for generating a query that instructs generation of at least one question sentence which asks about a matter serving as a basis for determining a degree of nursing care required; a generation control means for inputting, into a generative model that has been trained by machine learning to generate a question sentence in accordance with an input query, the query which is generated by the query generating means, and causing the generative model to generate the at least one question sentence; a presentation means for presenting, to a target person, the at least one question sentence that has been generated by the generative model; and a recording means for recording an answer of the target person to the at least one question sentence. This care needs assessment support program makes it possible to support determination of a degree of nursing care required.

### (Flow of Care Needs Assessment Support Method)

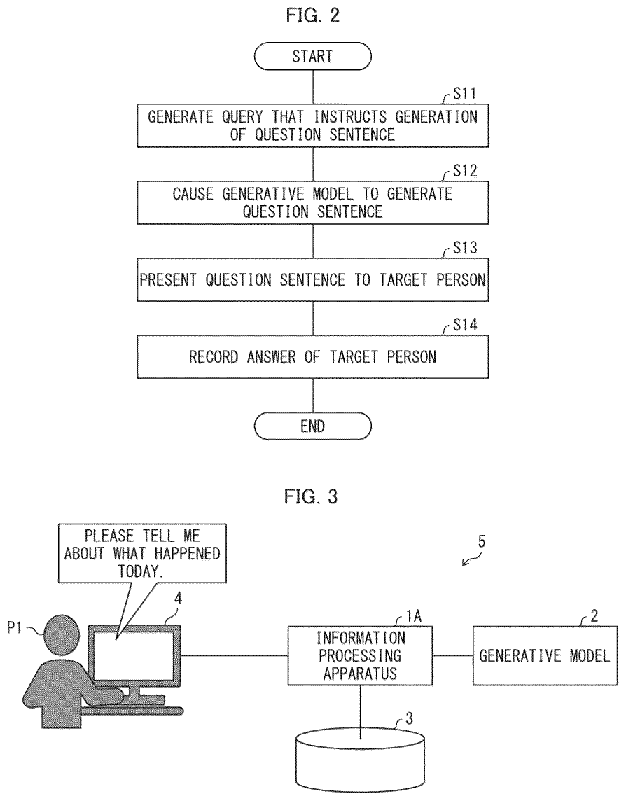

The following description will discuss, with reference to FIG. 2, a flow of a care needs assessment support method in accordance with the present example embodiment. FIG. 2 is a flowchart illustrating the flow of the care needs assessment support method. Note that steps of the care needs assessment support method may be carried out by a processor of the information processing apparatus 1 or by a processor of another apparatus. Alternatively, the steps may be carried out by processors provided in respective different apparatuses.

In S11 (a query generating process), at least one processor generates a query that instructs generation of a question sentence which asks about a matter serving as a basis for determining a degree of nursing care required.

In S12 (a generation control process), the at least one processor inputs, into a generative model that has been trained by machine learning to generate a question sentence in accordance with an input query, the query which has been generated in S11, and causes the generative model to generate the question sentence.

In S13 (a presentation process), the at least one processor presents, to a target person, the question sentence that has been generated by the generative model in S12.

In S14 (a recording process), the at least one processor records an answer of the target person to the question sentence that has been presented in S13.

As described above, a configuration is employed such that a care needs assessment support method in accordance with the present example embodiment includes: a query generating process for generating a query that instructs generation of at least one question sentence which asks about a matter serving as a basis for determining a degree of nursing care required; a generation control process for inputting, into a generative model that has been trained by machine learning to generate a question sentence in accordance with an input query, the query which has been generated in the query generating process, and causing the generative model to generate the at least one question sentence; a presentation process for presenting, to a target person, the at least one question sentence that has been generated by the generative model; and a recording process for recording an answer of the target person to the at least one question sentence, the query generating process, the generation control process, the presentation process, and the recording process each being carried out by at least one processor. Thus, the care needs assessment support method in accordance with the present example embodiment makes it possible to support determination of a degree of nursing care required.

### Second Example Embodiment

The following description will discuss a second example embodiment, which is an example embodiment of the present invention, in detail with reference to the drawings. Note that the scope of application of techniques which are employed in the present example embodiment is not limited to the present example embodiment. That is, techniques which are employed in the present example embodiment can be employed also in the other example embodiments included in the present disclosure, provided that no particular technical problem occurs. Moreover, techniques which are indicated in the drawings referred to for describing the present example embodiment can be employed also in the other example embodiments included in the present disclosure, provided that no particular technical problem occurs.

### (Overview of Care Needs Assessment Support System)

An overview of a care needs assessment support system in accordance with the present example embodiment will be described with reference to FIG. 3. FIG. 3 is a view describing an overview of a care needs assessment support system 5. The care needs assessment support system 5 is a system for supporting care needs assessment and is applicable to medical and healthcare fields. The care needs assessment support system 5 illustrated in FIG. 3 includes an information processing apparatus 1A, a generative model 2, a storage apparatus 3, and a display apparatus 4. Note that the generative model 2 may be stored in the information processing apparatus 1A, or may be stored in any other apparatus that is not illustrated. The storage apparatus 3 may be incorporated in the information processing apparatus 1A or may be provided outside the information processing apparatus 1A. The following description will discuss an example in which care needs assessment is supported. The care needs assessment support system 5 can support various cases where a degree of nursing care required is determined. Thus, a “matter serving as a basis for determining a nursing care level” in the following description can be read as a “matter serving as a basis for determining a degree of nursing care required”.

The information processing apparatus 1A generates a query that instructs generation of a question sentence which asks about a matter serving as a basis for determining a nursing care level, and inputs the generated query into the generative model 2 so as to cause the generative model 2 to generate the question sentence. By displaying, on the display apparatus 4, the question sentence that has been generated by the generative model 2, the information processing apparatus 1A presents the question sentence to a target person P1 whose nursing care level is to be assessed, and records, in the storage apparatus 3, an answer of the target person P1 to the presented question sentence. Note that a person who is different from an assessment target person whose nursing care level is to be assessed and who understands an ordinary physical and mental state of the assessment target person may be caused to answer the presented question.

The generative model 2 is a trained model that has been trained by machine learning to generate a question sentence in accordance with an input query. In the present example embodiment, an example will be described in which the generative model 2 is a language model that has learned, by machine learning, an arrangement of components (such as words) of a sentence and an arrangement of sentences in text. Using the generative model 2, which is a language model, makes it possible to generate a question sentence in text format from a query in text format. Note that the generative model 2 which is in accordance with a format of an input query and a format of a question sentence to be output need only be used as appropriate and is not limited to a language model. For example, in a case where a question sentence including an image is desired to be generated, a generative model that has been trained to generate an image need only be applied as the generative model 2.

In the example of FIG. 3, a question sentence “Please tell me about what happened today.” addressed to the target person P1 displayed on the display apparatus 4. This question sentence is generated by the generative model 2 on the basis of the query generated by the information processing apparatus 1A, and is a question sentence which asks about an ordinary life of the target person P1, the ordinary life serving as a basis for determining a nursing care level. The target person P1 inputs an answer to the question sentence into the information processing apparatus 1A, and the information processing apparatus 1A records the input answer in the storage apparatus 3.

Note that the answer may be input by any method. For example, the answer of the target person P1 may be input via a microphone or the like (not illustrated), or may be input via an input apparatus such as a keyboard (not illustrated). In a case where the answer is input by voice, the information processing apparatus 1A may record the input answer as voice data, or may convert the answer into text data and record the text data.

As described above, the care needs assessment support system 5 makes it possible to collect and record, without assistance from a survey staff member, the answer of the target person P1, the answer serving as a basis for determining a nursing care level of the target person P1. This makes it possible to reduce efforts to collect information serving as a basis for determining a nursing care level. Further, the care needs assessment support system 5 makes it possible to collect and record the answer of the target person P1 without assistance from a survey staff member, and thus makes it possible to determine an appropriate nursing care level on the basis of a straightforward answer of the target person.

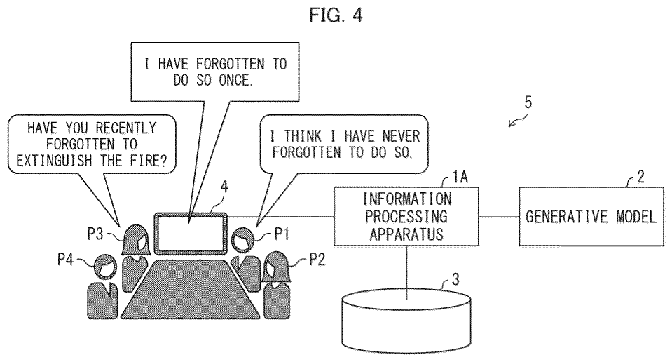

Further, the care needs assessment support system 5 can also support a visiting survey for assessment of a nursing care level. This will be described with reference to FIG. 4. FIG. 4 is a view describing support for a visiting survey by the care needs assessment support system 5. Note that the visiting survey is a survey such that a survey staff member visits a home or the like of a target person whose nursing care level is to be assessed, and carries out a hearing on a physical and mental state of the target person.

In the example of FIG. 4, survey staff members P3 and P4 visit a home of the target person P1 and a person P2 living with the target person P1, and carry out an oral survey for assessment of a nursing care level. More specifically, the survey staff member P3 asks the target person P1 whether the target person P1 has recently forgotten to extinguish the fire. To this question, the target person P1 answers that the target person P1 has not forgotten to extinguish the fire. Further, the answer to the above question asked by the survey staff member P3 is also displayed on the display apparatus 4. The answer displayed on the display apparatus 4 is generated by the information processing apparatus 1A, and indicates that the target person P has forgotten to extinguish the fire once.

Although details will be described later, the answer displayed on the display apparatus 4 is generated by the generative model 2 on the basis of the answer of the target person P1 which answer is recorded in the storage apparatus 3. This enables the survey staff members P3 and P4 to accurately determine the state of the target person P1 by referring to both an actual answer of the target person P1 and the answer displayed on the display apparatus 4.

For example, in a case where an answer of the target person P1 which answer has been obtained from a face-to-face oral survey matches the answer displayed on the display apparatus 4, the survey staff members P3 and P4 can determine that content of the answer of the target person P1 which answer has been obtained from the face-to-face oral survey is reliable. In contrast, as in the example of FIG. 4, in a case where the answer of the target person P1 which answer has been obtained from the face-to-face oral survey is different from the answer displayed on the display apparatus 4, it can be determined that an answer different from the fact may have been made due to a false memory or pride of the target person P1.

A visiting survey for assessment of a nursing care level is carried out in an unusual state in which a survey staff member visits a home of the target person P1, and time for the visiting survey is also limited. Thus, it is sometimes difficult to accurately understand an ordinary state of the target person P1 only from an utterance(s) of the target person P1 and/or the person P2 living with the target person P1. For example, in the example of FIG. 4, in a case where the answer of the target person P1 is different from the fact, an assessment result may be less severe than an actual state without using the care needs assessment support system 5.

In this regard, the care needs assessment support system 5 makes it possible to determine an appropriate nursing care level with use of an answer of the target person P1 which answer has been collected without assistance from a survey staff member and which is recorded in the storage apparatus 3. Further, since the visiting survey can be carried out by practically using the answer of the target person P1 which answer is recorded in the storage apparatus 3, the physical and mental state of the target person P1 can be more accurately reflected in a visiting survey result.

Further, also in a care needs assessment committee that finally determines assessment of a nursing care level, the care needs assessment support system 5 can be used as in the case of the foregoing visiting survey. That is, in the care needs assessment committee, an assessment committee member who determines a nursing care level need only ask the care needs assessment support system 5 a question about a matter that is desired to be checked for the target person P1. The care needs assessment support system 5 can cause the generative model 2 to generate an answer to the above question on the basis of the answer of the target person P1 which answer is recorded in the storage apparatus 3, and can present the generated answer to the assessment committee member.

Although details will be described later, the care needs assessment support system 5 can also generate a survey report that summarizes a result of the oral survey carried out with respect to the target person P1. In the care needs assessment committee, using this survey report to determine a nursing care level makes it possible to determine an appropriate nursing care level.

As described above, the care needs assessment support system 5 makes it possible to support determination of a nursing care level in accordance with the current mechanism for assessment of a nursing care level. Note that the care needs assessment support system 5 need only support collection of information serving at least as a basis for determining a nursing care level of the target person P1. Further, as described above, the care needs assessment support system 5 may support each of the following three stages: (i) hearing of an ordinary physical and mental state of the target person P1 prior to the visiting survey; (ii) hearing in the visiting survey; and (iii) the care needs assessment committee. This makes it possible to contribute to making the entire process of care needs assessment more efficient and more accurate.

### (Configuration of Information Processing Apparatus 1A)

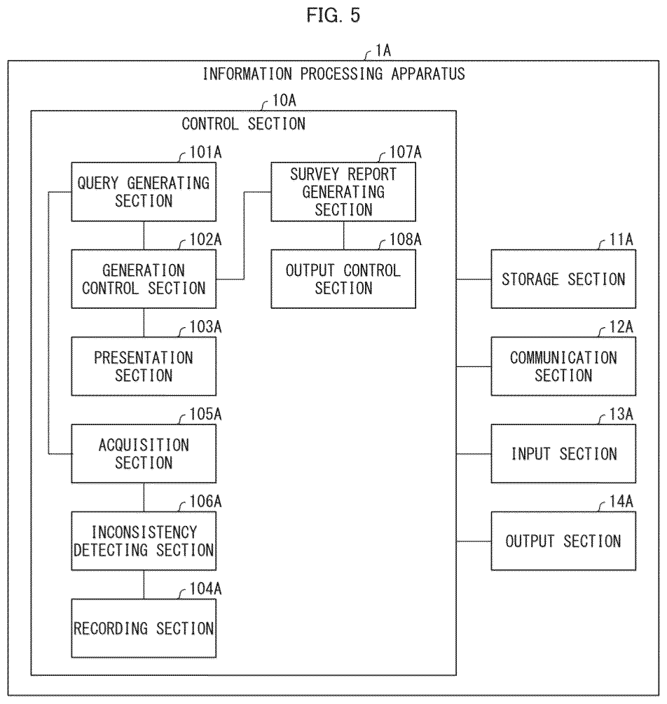

A configuration of an information processing apparatus 1A in accordance with the present example embodiment will be described below with reference to FIG. 5. FIG. 5 is a block diagram illustrating the configuration of the information processing apparatus 1A. Note that the information processing apparatus 1A may be an apparatus whose main function is care needs assessment support, or may be a general-purpose apparatus which includes other functions. The information processing apparatus 1A may be a stationary apparatus or a portable apparatus.

As illustrated in FIG. 5, the information processing apparatus 1A includes a control section 10A that collectively controls sections of the information processing apparatus 1A and a storage section 11A that stores various kinds of data used by the information processing apparatus 1A. The information processing apparatus 1A further includes a communication section 12A that allows the information processing apparatus 1A to communicate with another apparatus, an input section 13A that receives input to the information processing apparatus 1A, and an output section 14A that allows the information processing apparatus 1A to output data. The control section 10A includes a query generating section 101A, a generation control section 102A, a presentation section 103A, a recording section 104A, an acquisition section 105A, an inconsistency detecting section 106A, a survey report generating section 107A, and an output control section 108A.

As in the case of the query generating section 101 of the first example embodiment, the query generating section 101A generates a query that instructs generation of a question sentence which asks about a matter serving as a basis for determining a nursing care level. A method in which the query is generated by the query generating section 101A will be described in the “Method for generating query” section described later.

As in the case of the generation control section 102 of the first example embodiment, the generation control section 102A inputs, into the generative model 2 that has been trained by machine learning to generate a question sentence in accordance with an input query, the query which is generated by the query generating section 101A, and causes the generative model 2 to generate the question sentence. As described earlier, since the generative model 2 is a language model, the generation control section 102A inputs, into the generative model 2, a query described in natural language, and causes the generative model 2 to generate a question sentence described in natural language.

The generative model 2 may be a language model that has been trained, by machine learning, to learn one or both of (i) a question asked by a survey staff member who carries out a survey for assessment of a nursing care level and (ii) a question asked by an assessment committee member of a care needs assessment committee. The information processing apparatus 1A using such a generative model 2 brings about not only the effect brought about by the information processing apparatus 1 but also an effect of making it possible to present a question(s) similar to that/those asked by the survey staff member and/or the assessment committee member.

As in the case of the presentation section 103 of the first example embodiment, the presentation section 103A presents, to a target person, the question sentence that has been generated by the generative model 2. An answer may be presented in any manner. For example, the presentation section 103A may present an answer to the target person by displaying the answer on the display apparatus 4 illustrated in each of FIGS. 3 and 4. Alternatively, the presentation section 103A may cause the output section 14A to output the answer.

As in the case of the recording section 104 of the first example embodiment, the recording section 104A records an answer of the target person to the question sentence. The answer may be recorded anywhere. For example, the recording section 104A may record the answer in the storage apparatus 3 illustrated in each of FIGS. 3 and 4 or in the storage section 11A.

The acquisition section 105A acquires an answer of the target person to the question sentence presented by the presentation section 103A. A method in which the answer is acquired is not particularly limited. For example, the acquisition section 105A may acquire the answer input via the input section 13A or may acquire the answer input via an input apparatus external to the information processing apparatus 1A. As described earlier, the answer may be input by voice or may be input in text form. Further, the acquisition section 105A can similarly acquire questions from a survey staff member of a visiting survey, an assessment committee member of a care needs assessment committee, and the like.

The inconsistency detecting section 106A detects inconsistency in the answer of the target person to the question sentence that has been presented by the presentation section 103A. The information processing apparatus 1A including the inconsistency detecting section 106A brings about not only the effect brought about by the information processing apparatus 1 but also an effect of making it possible to obtain information useful in determining a cognitive function of a target person, i.e., whether the target person has made an inconsistent answer.

Note here that “inconsistency” means incoherence. A method for detecting inconsistency is not particularly limited. For example, in a case where content of the answer of the target person is inconsistent with the objective fact, the inconsistency detecting section 106A may determine that inconsistency has been detected. As a specific example, in a case where a date answered to a question asking about a birth date of a target person does not match a birth date of the target person, the inconsistency detecting section 106A may determine that inconsistency has been detected. Note that the birth date of the target person need only be input in advance by a person who has been found to have no problem with a cognitive function, such as a person living with the target person.

Further, the inconsistency detecting section 106A may detect inconsistency with use of data supporting an activity of the target person, such as a captured image of the target person, information detected by a mobile apparatus or a wearable apparatus possessed by the target person (e.g., position information, a call log, vital data such as a heart rate, an activity level, etc.), and/or detection values from various sensors (e.g., a human sensor) provided in an activity area of the target person. For example, in a case where it has been detected by a result of analysis of the captured image of the target person that the target person has fallen, but an answer of the target person to a question asking whether the target person has fallen is that the target person has not fallen, the inconsistency detecting section 106A may determine that inconsistency has been detected.

Further, the inconsistency detecting section 106A may detect inconsistency in answers to a respective plurality of questions to the target person. For example, in a case where questions identical in content to those asked in the past are asked again, and answers to the respective questions do not match each other in content, the inconsistency detecting section 106A may determine that inconsistency has been detected. Note that a method for determining whether the answers match each other in content is not particularly limited. For example, the inconsistency detecting section 106A may generate a query that includes a plurality of answers for which it is to be determined whether the answers match each other in content and that requires an answer as to whether the answers match each other in content, and may input the query into the language model 2. This allows the inconsistency detecting section 106A to determine, on the basis of an output of the language model 2, whether the input answers match each other in content.

The survey report generating section 107A uses the answer of the target person which answer is recorded in the storage apparatus 3 to generate a survey report showing a result of an oral survey for assessment of a nursing care level. The information processing apparatus 1A including the survey report generating section 107A brings about not only the effect brought about by the information processing apparatus 1 but also an effect of making it possible to save or reduce the labor required for generation of the survey report by the survey staff member. Note that details of a method for generating the survey report will be described in the “Method for generating survey report” section described later.

The output control section 108A causes an output apparatus to output the survey report generated by the survey report generating section 107A. A manner in which the survey report is output may be display output or may be print output. The output apparatus that outputs the survey report may be included in the information processing apparatus 1A or may be an apparatus external to the information processing apparatus 1A.

### (Method for Generating Query)

A method in which the query is generated by the query generating section 101A will be described. Any method is applicable to the query generating section 101A provided that the method makes it possible to generate a query that instructs generation of a question sentence which asks about a matter serving as a basis for determining a nursing care level.

For example, the query generating section 101A may generate the query that includes a document describing a survey item for determining a nursing care level and that instructs generation of a question on the basis of the document. This brings about not only the effect brought about by the information processing apparatus 1 but also an effect of making it possible to generate a question sentence in accordance with a survey item and description content that are described in the document.

For example, the query generating section 101A may generate a query including a document stating that a survey of whether it is possible to express an intension, whether it is possible to correctly answer a birth date or age, and the like is to be carried out for a cognitive function, which is one of survey items for determining a nursing care level. In this case, the query generating section 101A may generate a query including, for example, (i) a sentence “Please generate, on the basis of the following document, a question sentence for checking a cognitive function of a target person.” and (ii) the document. Note that the sentence and the document which are included in the query need only be stored in advance in the storage section 11A or the like.

Further, by carrying out a similar process for each survey item, the query generating section 101A can generate a query that causes a question sentence for each survey item to be generated. Note that it is not essential to generate a query for each survey item. The query generating section 101A may generate a query that instructs generation of a question about a plurality of survey items. Further, in the case where the document has a large volume, the query generating section 101A may generate a query that instructs generation of a question by summarization of content of the document.

The query generating section 101A may also generate a query including information pertaining to the target person. This makes it possible to generate a question in accordance with the target person. For example, the query generating section 101A may generate a query including age, gender, a medical history, etc. of the target person. This makes it possible to generate a question in consideration of age, gender, a medical history, etc. of the target person.

The query generating section 101A preferably generates a query which specifies that a question to be generated is a question for a survey to determine a nursing care level. For example, the query generating section 101A may generate a query including a sentence such as “Please generate a question for a survey to determine a nursing care level.” or “Please generate a question to present to a target person as a survey staff member who determines a nursing care level.”.

The query generating section 101A may also generate a query including a specific example of a question to be generated. This makes it possible to generate a question in accordance with a specific example. For example, the query generating section 101A may generate a query including a sentence such as “Please generate a question with reference to the following specific example. Specific example: Regarding a cognitive function, have you ever been unable to come home from where you have gone to?”. Note that two or more specific examples may be included. Note also that a question actually asked by a survey staff member or an assessment committee member may be included as a specific example.

Further, the presentation section 103A may sequentially present a generated plurality of questions to the target person with time intervals apart, instead of collectively presenting the generated plurality of questions at a time, and the acquisition section 105A may sequentially acquire answers to the presented questions. In this case, the generation control section 102A may input, into the generative model 2, an answer of the target person to a presented question sentence, and cause the generative model 2 to generate a new question sentence related to the question sentence. This brings about not only the effect brought about by the information processing apparatus 1 but also an effect of making it possible to generate a new question sentence that is based on the answer of the target person.

For example, the query generating section 101A may generate a query that includes an answer of the target person and that instructs generation of a question sentence which is based on the answer. The generation control section 102A inputs this query into the generative model 2, so that a new question sentence which is based on the answer of the target person can be generated. For example, by inputting, into the generative model 2, a query that instructs generation of a question sentence based on an answer which has been made to a question asking whether the target person has forgotten to extinguish the fire and which says that the target person has forgotten to extinguish the fire, it is also possible to generate a question sentence asking a frequency at which the target person forgets to extinguish the fire. Further, for example, in a case where it is impossible to make the target person understand content of a question, it is possible to generate a more simply expressed question sentence. Alternatively, in a case where the target person has been displeased with the content of the question, it is possible to, for example, generate a question sentence with a manner in which the question is asked changed.

Note here that matters about which the target person is to be asked are diverse in an oral survey for determining a nursing care level. It is therefore assumed that collectively presenting, at a time, questions about all matters to be checked results in a greater load on the target person who is to answer the questions.

Thus, the presentation section 103A preferably decentralizes or divides, in a period up to a time point at which an oral survey is to be ended, a presentation timing at which a generated plurality of question sentences are presented. This brings about not only the effect brought about by the information processing apparatus 1 but also an effect of making it possible to minimize a load on the target person who answers the questions. Note here that decentralizing or dividing a presentation timing in a period means determining the presentation timing so that a presentation timing at which a question is presented is equally or substantially equally divided in the period, or means determining the presentation timing so that a presentation timing at which a question is presented is not temporarily concentrated. Note that the presentation section 103A may determine, in advance, a timing at which each question sentence is presented, or may present a subsequent question sentence on condition that a certain time period or longer has elapsed after previous presentation of the question sentence.

The time point at which the oral survey is to be ended need only be designated in advance. For example, a day before a day on which a visiting survey is to be carried out may be designated as the time point at which the oral survey is to be ended. In this case, a presentation timing at which a plurality of question sentences are presented is decentralized or divided in a period from a time point at which such designation was carried out to the day before the day on which the visiting survey is to be carried out. Note that the presentation timing may be determined in consideration of, for example, a schedule of the target person.

In a case where there is a sufficient period before the time point at which the oral survey is to be ended, the presentation section 103A may present an identical question sentence (or question sentences that share content) a plurality of times at different timings. In this case, it is possible to cause the inconsistency detecting section 106A to determine whether there is inconsistency in an answer to the identical question sentence.

The query generating section 101A may generate a query that designates a period in which a question sentence is presented and that instructs generation of a question group which is asked over the period. For example, in a case where the oral survey needs to be ended by one month later, the query generating section 101A may generate a query that instructs generation of a question group which allows each survey item described in an input document to be comprehensively checked in one month. This makes it possible to generate question sentences whose content and number are in accordance with the period in which a question sentence is presented.

### (Method for Generating Survey Report)

A method in which the survey report is generated by the survey report generating section 107A will be described. In general, a format of the survey report is predetermined. Thus, in accordance with the predetermined format, the survey s report generating section 107A need only extract necessary information from the answer of the target person which answer is recorded in the storage apparatus 3, and generate the survey report. For example, in a case where the survey report has an item describing a physical function of the target person, the survey report generating section 107A need only extract an answer to a question about the physical function from among answers of the target person which are recorded in the storage apparatus 3, and enter the answer in the item.

Alternatively, the survey report generating section 107A may use the generative model 2 to generate the survey report. For example, the survey report generating section 107A may generate a query that instructs extraction, from each of answers of the target person which are recorded in the storage apparatus 3, of a matter to be entered in each input item of the survey report. By inputting the generated query into the generative model 2, the survey report generating section 107A can specify the matter to be entered in each input item of the survey report, and generate the survey report. Note that the query may be generated by the query generating section 101A and that the query may be input into the generative model 2 by the generation control section 102A.

The survey report generated by the survey report generating section 107A may be used, as it is, as one of bases for determining a care needs assessment. Alternatively, with reference to the survey report generated by the survey report generating section 107A, the survey staff member may prepare a formal survey report after the visiting survey. In the latter case, the survey report generating section 107A may generate information useful in the visiting survey, and cause the output control section 108A or the presentation section 103A to present the information to the survey staff member.

Examples of the information useful in the visiting survey include a rate of checking of each of the plurality of survey items. Assume, for example, that, among three survey items, i.e., a physical function, a vital function, and a cognitive function, an insufficient number of question sentences s about the vital function have been presented, or the number of answers to the question sentences about the vital function is insufficient. In such a case, the survey report generating section 107A may generate information indicating that a rate of checking of the vital function is low or that the vital function should be sufficiently checked in the visiting survey. The information may be represented by a sentence or may be represented by an image such as a graph. For example, the survey report generating section 107A may cause the presentation section 103A to present a radar chart showing the rate of checking of each of the plurality of survey items. This allows the rate of checking of each of the plurality of survey items to be recognized at a glance.

Note that which question corresponds to which of the survey items may be determined by the generative model 2 or another language model. Further, in a case where question sentences are generated for the respective survey items, recording a corresponding survey item in association with each of the generated question sentences makes it possible to specify a correspondence between a question sentence and a survey item.

In a case where there is an imbalance in the rate of checking of each of the survey items, in generating, next time or later, a query that instructs generation of a question sentence, the query generating section 101A may generate a query that urges the rate of checking of each of the survey items to be in a proper range. For example, the query generating section 101A may generate a query that requests generation of a question sentence so that the survey items can be equally checked, or may generate a query that requests intensive generation of a question sentence for a survey item whose rate of checking is low.

The information processing apparatus 1A may include a model updating section which updates the generative model 2 so that a proper quantity and quality of question sentences will be generated for a survey item which has been insufficiently checked. For example, the model updating section may update the generative model 2 by fine-tuning the generative model 2 by using, as training data, an example of a question about the survey item that has been insufficiently checked. Note that the training data need only be input by a user or the like of the information processing apparatus 1A.

(Method for Generating Answer to Question)

The following description will discuss a method for generating an answer to a question from a survey staff member in a visiting survey or a question from an assessment committee member in a care needs assessment committee. Since the method for generating an answer is similar regardless of whether a questioner is the survey staff member or the assessment committee member, the following description will discuss an example in which the questioner is the survey staff member.

The question from the survey staff member is acquired by the acquisition section 105A. In a case where the acquisition section 105A has acquired the question, the query generating section 101A generates a query that instructs generation of an answer (which can also be rephrased as an answer sentence) to the acquired question. More specifically, on the basis of an answer recorded in the storage apparatus 3 by an oral survey by the care needs assessment support system 5, the query generating section 101A generates a query that instructs generation of an answer to the question acquired by the acquisition section 105A.

For example, the query generating section 101A may generate a query including (i) each answer read from the storage apparatus 3, (ii) the question acquired by the acquisition section 105A, and (iii) a template for a generation instruction that specifies a generation method. The template may be, for example, a sentence such as “Please generate an answer to an input question on the basis of each input answer.”.

The generation control section 102A inputs, into the generative model 2, the query thus generated. This allows the generative model 2 to generate an answer that is made to the question from the survey staff member and that is based on the answer recorded in the storage apparatus 3. Note that the answer to the question from the survey staff member may be generated by another language model (that has been trained by machine learning to generate an answer to a question) other than the generative model 2.

As described above, the generation control section 102A causes a language model that has been trained by machine learning to generate an answer to a question to generate an answer which is to a question from a survey staff member who carries out a survey for assessment of a nursing care level or from an assessment committee member of a care needs assessment committee and which is based on the recorded answer. This brings about not only the effect brought about by the information processing apparatus 1 but also an effect of making it possible to obtain information useful for a survey by the survey staff member or information useful for assessment in the care needs assessment committee. For example, as described with reference to

FIG. 4, in a visiting survey, it is also possible to ask both the target person and the information processing apparatus 1A for the answer to the question asked by the survey staff member. This allows the survey staff member to obtain an appropriate survey result in consideration of answers made by both the target person and the information processing apparatus 1A.

Further, conventionally, the care needs assessment committee that the target person does not attend has no choice but to assess a nursing care level on the basis of submitted documents such as a survey report and a physician's written opinion. In a case where any unclear point is found in the submitted documents, it is difficult to ask the target person about the unclear point. In this regard, using the care needs assessment support system 5 makes it possible to present an answer that is made to a question about an unclear point and that is based on the recorded answer. This makes it possible to carry out proper assessment after clarifying the unclear point.

### (Another Method for Using Answer to Question)

The answer recorded in the storage apparatus 3 can be used in an application other than assessment of a nursing care level. For example, the information processing apparatus 1A may include a recommendation section that, on the basis of the answer of the target person which answer is recorded in the storage apparatus 3, determines a product or service to be recommended to the target person. It is only necessary to determine in advance what product or service to recommend to what answer. For example, it may be determined in advance that, to an answer stating that a joint is difficult to move, a physician, a clinic, or the like that has rich experience in coping with such a symptom is to be recommended. In this case, the recommendation section determines that the physician, the clinic, or the like is to be recommended to the target person who has answered that a joint is difficult to move. The product or service determined by the recommendation section need only be presented to the target n by the presentation section 103A.

On condition that consent of the target person has been obtained, the information processing apparatus 1A may provide a medical worker such as a primary care physician of the target person with the answer of the target person which answer is recorded in the storage apparatus 3. This allows the medical worker to understand an ordinary state of the target person, and can be helpful in providing an appropriate and smooth medical service.

Further, even after the visiting survey has been ended, the information processing apparatus 1A may continue to present a question to the target person and to collect and record an answer. In this case, the information processing apparatus 1A may include a state change detecting section that detects a change in physical or mental state of the target person on the basis of a change over time in content of the recorded answer. This allows the change in physical or mental state of the target person to be less likely to be overlooked.

For example, by comparing answer groups of the target person to question groups that have been presented for each certain period of time and that share content, the state change detecting section may determine whether the state has changed. Note that whether a change in content of answers has occurred can be determined by, for example, comparing, respective feature vectors of the answers, or can be determined with use of the generative model 2 or another language model. In a case where the state change detecting section detects a change in state, the change may be notified to the target person by the presentation section 103A. The change in state may be notified to not only the target person but also, for example, a family of the target person, a medical worker, a care service provider, or the like.

### (Flow of Process: Prior to Visiting Survey)

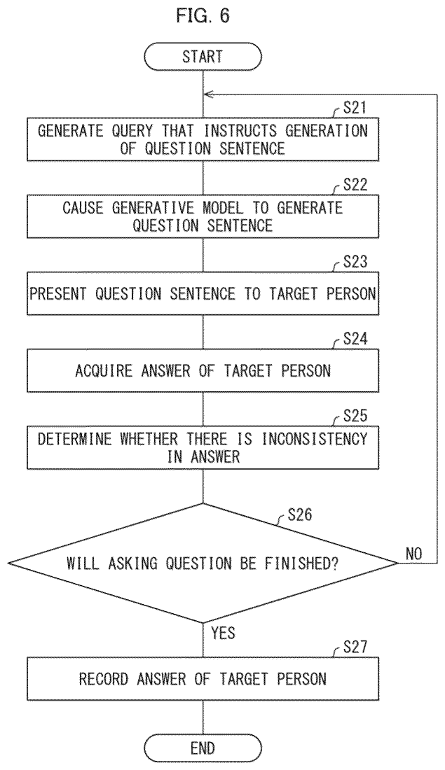

A flow of a process carried out by the information processing apparatus 1A will be described below with reference to FIG. 6. FIG. 6 is a flowchart illustrating the flow of the process carried out by the information processing apparatus 1A. This process is carried out by, for example, prior to a visiting survey by a survey staff member. The flow of FIG. 6 includes steps of the care needs assessment support method in accordance with the present example embodiment.

In S21 (a query generating process), the query generating section 101A generates a query that instructs generation of a question sentence which asks about a matter serving as a basis for determining a nursing care level. Since a method for generating the query is as described earlier, a description thereof is not repeated here.

In S22 (a generation control process), the generation control section 102A inputs, into the generative model 2 that has been trained by machine learning to generate a question sentence in accordance with an input query, the query which has been generated in S21, and causes the generative model 2 to generate the question sentence. Note that, in S22, the generation control section 102A may cause the generative model 2 to generate a plurality of question sentences.

In S23 (a presentation process), the presentation section 103A presents, to a target person, the question sentence that has been generated by the generative model 2. Note that, in a case where the plurality of question sentences have been generated in S22, the presentation section 103A may present a part of the generated question sentences. In that case, if a determination result in S26 described later is NO, the process transitions to S23, and the presentation section 103A presents another part of the generated plurality of question sentences. By repeating such a process, the generated plurality of question sentences can be sequentially presented to the target person. The process in S23 need not necessarily be carried out immediately after the process in S21 and S22. For example, the presentation section 103A may present, in a case where the target person carries out an input operation to request the information processing apparatus 1A to present a question, the question sentence(s) generated in advance by the query generating section 101A and the generation control section 102A.

In S24, the acquisition section 105A acquires an answer of the target person to the question sentence presented in S23. As described earlier, the acquisition section 105A may acquire the answer that is input via the input section 13A, or may acquire the answer that is input via an input apparatus external to the information processing apparatus 1A. Further, the answer may be input by voice or may be input in text form.

In S25, the inconsistency detecting section 106A determines whether there is inconsistency in the answer acquired in S24. Since a method for determining presence or absence of inconsistency is as described earlier, a description thereof is not repeated here. Further, a timing at which presence or absence of inconsistency is determined is not limited to the example of FIG. 6. For example, presence or absence of inconsistency may be determined after the process in S26 or after the process in S27.

In S26, the query generating section 101A determines whether to finish asking a question to the target person. A condition for finishing asking the question need only be determined in advance. Asking the question may be finished under, for example, a condition that the number of questions of each survey item reaches a predetermined number. Alternatively, for example, in consideration of fatigue of the target person, asking the question may be finished under a condition that at least a certain time period (e.g., 15 minutes) has elapsed since the process of FIG. 6 has been started or that a total number of presented questions has reached at least a predetermined number (e.g., 10).

In a case where the determination result in S26 is YES, the process proceeds to S27. In contrast, in a case where the determination result in S26 is NO, the process returns to S21, and the query generating section 101A generates a query that instructs generation of a new question sentence. In so doing, the query generating section 101A may generate the query including the answer acquired in S24. This makes it possible to generate the new question sentence that is based on the answer of the target person.

In S27 (a recording process), the recording section 104A records the answer acquired in S24, i.e., the answer of the target person to the question sentence presented in S23. This ends the process of FIG. 6. Note that the answer may be recorded every time the answer is acquired.

### (Flow of Process: Generation of Survey Report)

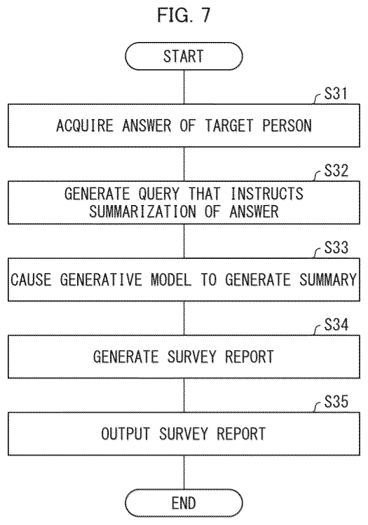

A flow of a process in which the information processing apparatus 1A generates a survey report will be described below with reference to FIG. 7. FIG. 7 is a flowchart of the process in which the information processing apparatus 1A generates the survey report.

In S31, the acquisition section 105A acquires an answer of the target person which answer is recorded in the storage apparatus 3 (more accurately, a plurality of answers recorded during a period of the oral survey by the care needs assessment support system 5).

In S32, the query generating section 101A generates a query that instructs summarization of the answer acquired in S31. For example, the query generating section 101A may generate a query including (i) a template that instructs summarization of the answer, such as “Please summarize the following answer.” and (ii) each answer acquired in S31. Further, for example, the query generating section 101A may generate a query that includes each item to be entered in the survey report and that instructs generation of a summary to be entered in the each item.

In S33, the generation control section 102A causes the generative model 2 to generate a summary of a plurality of answers to a plurality of question sentences. Specifically, the generation control section 102A inputs, into the generative model 2, the query generated in S32, and thus the generative model 2 generates the summary. Note that the summary may be alternatively generated by a language model other than the generative model 2.

In S34, the survey report generating section 107A uses the summary generated in S33 to generate a survey report that summarizes a result of the oral survey by the care needs assessment support system 5. For example, in a case where a summary of answers of the target person for each item to be entered in the survey report has been generated, the survey report generating section 107A can generate the survey report by entering the summary of the answers in a corresponding item.

In S35, the output control section 108A outputs the survey report generated in S34. This ends the process of FIG. 7. Note that the information processing apparatus 1A may be configured to accept modification by a survey staff member to the survey report output in S35.

### (Flow of Process: Visiting Survey/Care Needs Assessment Committee)

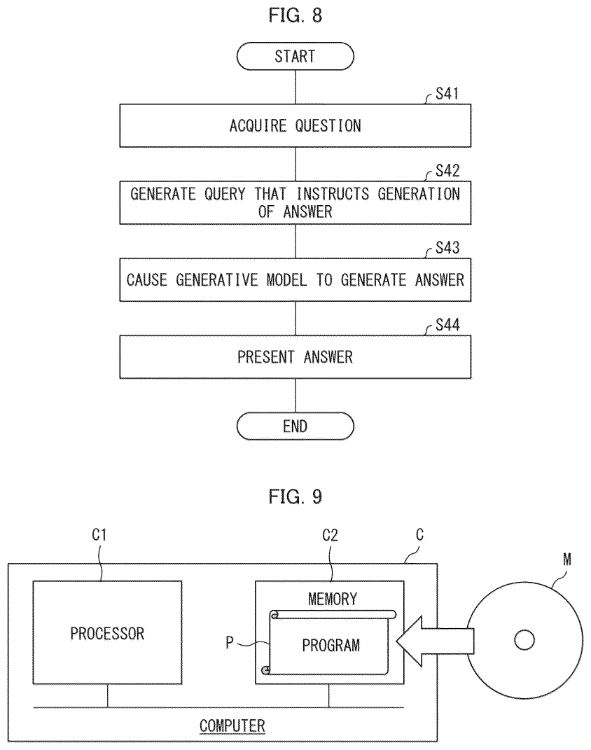

A flow of a process carried out by the information processing apparatus 1A in a visiting survey or a care needs assessment committee will be described below with reference to FIG. 8. FIG. 8 is a flowchart of the process carried out by the information processing apparatus 1A in the visiting survey or the care needs assessment committee. The following description will discuss a process carried out in the visiting survey. Note that the process carried out in the care needs assessment committee is similar except that a person who asks a question and to whom an answer to the question is to be presented is changed from the survey staff member to an assessment committee member.

In S41, the acquisition section 105A acquires a question from the survey staff member. Further, the question from the survey staff member may be input by voice or may be input in text form. In a case where the question has been input by voice, the acquisition section 105A converts the input voice into text.

In S42, the query generating section 101A generates a query that instructs generation of an answer to the question acquired in $41. More specifically, on the basis of an answer recorded in the storage apparatus 3 by an oral survey by the care needs assessment support system 5, the query generating section 101A generates a query that instructs generation of an answer to the question acquired in S41.

In S43, the generation control section 102A causes the generative model 2 to generate the answer to the question acquired in S41. Specifically, the generation control section 102A inputs, into the generative model 2, the query generated in S42, and thus the generative model 2 generates the answer. Note that the answer to the question from the survey staff member may be alternatively generated by a language model other than the generative model 2.

In S44, the presentation section 103A presents, to the survey staff member, the answer generated in S43. This ends the process of FIG. 8. Note that the answer may be presented in S44 by audio output or by display output on the display apparatus 4 or the like.

Further, the information processing apparatus 1A may accept another question related to the answer presented in S44. In that case, the process in S41 to S44 is repeated again. In the process in S42 at the second time and later, the query generating section 101A may generate a query including the answer presented in S44. This makes it possible to generate an answer that is based on the context of dialogue such that a new question has been addressed to the answer presented earlier.

### Variation

The processes described in the foregoing example embodiments may be carried out by any subject, which is not limited to the foregoing examples. For example, functions similar to those of the information processing apparatus 1, 1A can be realized by a plurality of apparatuses that can communicate with each other. The processes shown in the flowcharts illustrated in FIGS. 6 to 8 may be carried out by a single apparatus (that can be rephrased as a processor) or by a plurality of apparatuses (that can be rephrased also as processors).

### Software Implementation Example

Some or all of the functions of the information processing apparatuses 1 and 1A can be realized by hardware such as an integrated circuit (IC chip), or can be realized by software. Note that the “functions” as used herein include not only functions realized by blocks illustrated in FIGS. 1 and 5 but also functions of, for example, the model updating section (model updating means), the recommendation section (recommendation means), and the state change detecting section (state change detecting means), which have been described earlier.

In a case where some or all of the functions of the information processing apparatuses 1 and 1A are realized by software, the information processing apparatuses 1 and 1A are each realized by, for example, a computer that executes instructions of a program that is software realizing the foregoing functions. FIG. 9 illustrates an example of such a computer (hereinafter referred to as “computer C”). FIG. 9 is a block diagram illustrating a hardware configuration of the computer C which functions as the information processing apparatus 1 or 1A.

The computer C includes at least one processor C1 and at least one memory C2. In the memory C2, a program (care needs assessment support program) P for causing the computer C to operate as the information processing apparatus 1 or 1A is recorded. In the computer C, the functions of the information processing apparatus 1 or 1A are realized by the processor C1 reading the program P from the memory C2 and executing the program P.

Examples of the processor C1 encompass a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, and a combination thereof. Examples of the memory C2 include a flash memory, a hard disk drive (HDD), a solid state drive (SSD), and a combination thereof.

Note that the computer C may further include a random access memory (RAM) in which the program P is loaded during execution of the program P and/or in which various kinds of data are temporarily stored. The computer C may further include a communication interface via which the computer C transmits and receives data to and from another apparatus. The computer C may further include an input/output interface via which the computer C is connected to an input/output apparatus(es) such as a keyboard, a mouse, a display, and/or a printer.

The program P can be stored in a non-transitory tangible recording medium M which is readable by the computer C. Examples of the recording medium M encompass a tape, a disk, a card, a semiconductor memory, and a programmable logic circuit. The computer C can acquire the program P via the recording medium M. The program P can be transmitted via a transmission medium. Examples of the transmission medium encompass communications network and a broadcast wave. The computer C can acquire the program P also via such a transmission medium.

The foregoing functions of the information processing apparatuses 1 and 1A may be realized by a single processor provided in a single computer, may be realized by cooperation by a plurality of processors provided in a single computer, or may be realized by cooperation by a plurality of processors provided in a respective plurality of computers. A program for causing the information processing apparatuses 1 and 1A to realize the foregoing functions may be stored in a single memory provided in a single computer, may be stored dispersedly in a plurality of memories provided in a single computer, or may be stored dispersedly in a plurality of memories provided in a respective plurality of computers.

### ADDITIONAL REMARKS

The present disclosure includes techniques described in supplementary notes below. Note, however, that the present invention is not limited to the techniques described in the supplementary notes below, but may be altered in various ways by a skilled person within the scope of the claims.

### Additional Remark A

### (Supplementary Note A1)

An information processing apparatus including: a query generating means for generating a query that instructs generation of at least one question sentence which asks about a matter serving as a basis for determining a degree of nursing care required; a generation control means for inputting, into a generative model that has been trained by machine learning to generate a question sentence in accordance with an input query, the query which is generated by the query generating means, and causing the generative model to generate the at least one question sentence; a presentation means for presenting, to a target person, the at least one question sentence that has been generated by the generative model; and a recording means for recording an answer of the target person to the at least one question sentence.

### (Supplementary Note A2)

The information processing apparatus described in supplementary note A1, wherein the generative model is a language model that has been trained, by machine learning, to learn one or both of (i) a question asked by a survey staff member who carries out a survey for assessment of a nursing care level and (ii) a question asked by an assessment committee member of a care needs assessment committee.

### (Supplementary Note A3)

The information processing apparatus described in supplementary note A1 or A2, wherein the generation control means inputs, into the generative model, the answer of the target person to the at least one question sentence that has been presented, and causes the generative model to generate a new question sentence related to the at least one question sentence.

### (Supplementary Note A4)

The information processing apparatus described in any one of supplementary notes A1 to A3, further including a survey report generating means for using the recorded answer of the target person to generate a survey report showing a result of an oral survey for assessment of a nursing care level.

### (Supplementary Note A5)

The information processing apparatus described in any one of supplementary notes A1 to A4, wherein the query generating means generates the query that includes a document describing a survey item for determining a nursing care level and that instructs generation of a question on the basis of the document.

### (Supplementary Note A6)

The information processing apparatus described in any one of supplementary notes A1 to A5, wherein the presentation means decentralizes or divides, in a period up to a time point at which an oral survey is to be ended, a presentation timing at which a plurality of question sentences that the at least one question sentence comprises are presented.

### (Supplementary Note A7)

The information processing apparatus described in any one of supplementary notes A1 to A6, further comprising an inconsistency detecting means for detecting inconsistency in the answer of the target person to the at least one question sentence that has been presented.

### (Supplementary Note A8)

The information processing apparatus described in any one of supplementary notes A1 to A7, wherein the generation control means causes a language model that has been trained by machine learning to generate an answer to a question to generate an answer which is to a question from a survey staff member who carries out a survey for assessment of a nursing care level or from an assessment committee member of a care needs assessment committee and which is based on the recorded answer.

### Additional Remark B

### (Supplementary Note B1)

A care needs assessment support method including: a query generating process for generating a query that instructs generation of at least one question sentence which asks about a matter serving as a basis for determining a degree of nursing care required; a generation control process for inputting, into a generative model that has been trained by machine learning to generate a question sentence in accordance with an input query, the query which has been generated in the query generating process, and causing the generative model to generate the at least one question sentence; a presentation process for presenting, to a target person, the at least one question sentence that has been generated by the generative model; and a recording process for recording an answer of the target person to the at least one question sentence, the query generating process, the generation control process, the presentation process, and the recording process each being carried out by at least one processor.

### (Supplementary Note B2)

The care needs assessment support method described in supplementary note B1, wherein the generative model is a language model that has been trained, by machine learning, to learn one or both of (i) a question asked by a survey staff member who carries out a survey for assessment of a nursing care level and (ii) a question asked by an assessment committee member of a care needs assessment committee.

### (Supplementary Note B3)

The care needs assessment support method described in supplementary note B1 or B2, wherein the at least one processor inputs, into the generative model, the answer of the target person to the at least one question sentence that has been presented, and causes the generative model to generate a new question sentence related to the at least one question sentence.

### (Supplementary Note B4)

The care needs assessment support method described in any one of supplementary notes B1 to B3, further including a survey report generating process for using the recorded answer of the target person to generate a survey report showing a result of an oral survey for assessment of a nursing care level, the survey report generating process being carried out by the at least one processor.

### (Supplementary Note B5)

The care needs assessment support method described in any one of supplementary notes B1 to B4, wherein, in the query generating process, the at least one processor generates the query that includes a document describing a survey item for determining a nursing care level and that instructs generation of a question on the basis of the document.

### (Supplementary Note B6)

The care needs assessment support method described in any one of supplementary notes B1 to B5, wherein, in the presentation process, the at least one processor decentralizes or divides, in a period up to a time point at which an oral survey is to be ended, a presentation timing at which a plurality of question sentences that the at least one question sentence comprises are presented.

### (Supplementary Note B7)

The care needs assessment support method described in any one of supplementary notes B1 to B6, further including an inconsistency detecting process for detecting inconsistency in the answer of the target person to the at least one question sentence that has been presented, the inconsistency detecting process being carried out by the at least one processor.

### (Supplementary Note B8)

The care needs assessment support method described in any one of supplementary notes B1 to B7, wherein the at least one processor carries out a process for causing a language model that has been trained by machine learning to generate an answer to a question to generate an answer which is to a question from a survey staff member who carries out a survey for assessment of a nursing care level or from an assessment committee member of a care needs assessment committee and which is based on the recorded answer.

### Additional Remark C

### (Supplementary Note C1)

A care needs assessment support program for causing a computer to function as: a query generating means for generating a query that instructs generation of at least one question sentence which asks about a matter serving as a basis for determining a degree of nursing care required; a generation control means for inputting, into a generative model that has been trained by machine learning to generate a question sentence in accordance with an input query, the query which is generated by the query generating means, and causing the generative model to generate the at least one question sentence; a presentation means for presenting, to a target person, the at least one question sentence that has been generated by the generative model; and a recording means for recording an answer of the target person to the at least one question sentence.

### (Supplementary Note C2)

The care needs assessment support program described in supplementary note C1, wherein the generative model is a language model that has been trained, by machine learning, to learn one or both of (i) a question asked by a survey staff member who carries out a survey for assessment of a nursing care level and (ii) a question asked by an assessment committee member of a care needs assessment committee.

### (Supplementary Note C3)

The care needs assessment support program described in supplementary note C1 or C2, wherein the generation control means inputs, into the generative model, the answer of the target person to the at least one question sentence that has been presented, and causes the generative model to generate a new question sentence related to the at least one question sentence.

### (Supplementary Note C4)

The care needs assessment support program described in any one of supplementary notes C1 to C3, wherein the care needs assessment support program causes the computer to function as a survey report generating means for using the recorded answer of the target person to generate a survey report showing a result of an oral survey for assessment of a nursing care level.

### (Supplementary Note C5)

The care needs assessment support program described in any one of supplementary notes C1 to C4, wherein the query generating means generates the query that includes a document describing a survey item for determining a nursing care level and that instructs generation of a question on the basis of the document.

### (Supplementary Note C6)

The care needs assessment support program described in any one of supplementary notes C1 to C5, wherein the presentation means decentralizes or divides, in a period up to a time point at which an oral survey is to be ended, a presentation timing at which a plurality of question sentences that the at least one question sentence comprises are presented.

### (Supplementary Note C7)

The care needs assessment support program described in any one of supplementary notes C1 to C6, wherein the care needs assessment support program causes the computer to function as an inconsistency detecting means for detecting inconsistency in the answer of the target person to the at least one question sentence that has been presented.

### (Supplementary Note C8)

The care needs assessment support program described in any one of supplementary notes C1 to C7, wherein the generation control means causes a language model that has been trained by machine learning to generate an answer to a question to generate an answer which is to a question from a survey staff member who carries out a survey for assessment of a nursing care level or from an assessment committee member of a care needs assessment committee and which is based on the recorded answer.

### Additional Remark D

### (Supplementary Note D1)

An information processing apparatus including at least one processor, the at least one processor carrying out: a query generating process for generating a query that instructs generation of at least one question sentence which asks about a matter serving as a basis for determining a degree of nursing care required; a generation control process for inputting, into a generative model that has been trained by machine learning to generate a question sentence in accordance with an input query, the query which has been generated in the query generating process, and causing the generative model to generate the at least one question sentence; a presentation process for presenting, to a target person, the at least one question sentence that has been generated by the generative model; and a recording process for recording an answer of the target person to the at least one question sentence.

The information processing apparatus may further include a memory. The memory may store a program for causing the at least one processor to carry out each of the processes.

### (Supplementary Note D2)

The information processing apparatus described in supplementary note D1, wherein the generative model is a language model that has been trained, by machine learning, to learn one or both of (i) a question asked by a survey staff member who carries out a survey for assessment of a nursing care level and (ii) a question asked by an assessment committee member of a care needs assessment committee.

### (Supplementary Note D3)

The information processing apparatus described in supplementary note D1 or D2, wherein the at least one processor inputs, into the generative model, the answer of the target person to the at least one question sentence that has been presented, and causes the generative model to generate a new question sentence related to the at least one question sentence.

### (Supplementary Note D4)

The information processing apparatus described in any one of supplementary notes D1 to D3, wherein the at least one processor carries out a survey report generating process for using the recorded answer of the target person to generate a survey report showing a result of an oral survey for assessment of a nursing care level.

### (Supplementary Note D5)

The information processing apparatus described in any one of supplementary notes D1 to D4, wherein, in the query generating process, the at least one processor generates the query that includes a document describing a survey item for determining a nursing care level and that instructs generation of a question on the basis of the document.

### (Supplementary Note D6)

The information processing apparatus described in any one of supplementary notes D1 to D5, wherein, in the presentation process, the at least one processor decentralizes or divides, in a period up to a time point at which an oral survey is to be ended, a presentation timing at which a plurality of question sentences that the at least one question sentence comprises are presented.

### (Supplementary Note D7)

The information processing apparatus described in any one of supplementary notes D1 to D6, wherein the at least one processor carries out an inconsistency detecting process for detecting inconsistency in the answer of the target person to the at least one question sentence that has been presented.

### (Supplementary Note D8)

The information processing apparatus described in any one of supplementary notes D1 to D7, wherein the at least one processor carries out a process for causing a language model that has been trained by machine learning to generate an answer to a question to generate an answer which is to a question from a survey staff member who carries out a survey for assessment of a nursing care level or from an assessment committee member of a care needs assessment committee and which is based on the recorded answer.

### Additional Remark

### (Supplementary Note E1)

A non-transitory recording medium recording therein a care needs assessment support program for causing a computer to carry out: a query generating process for generating a query that instructs generation of at least one question sentence which asks about a matter serving as a basis for determining a degree of nursing care required; a generation control process for inputting, into a generative model that has been trained by machine learning to generate a question sentence in accordance with an input query, the query which has been generated in the query generating process, and causing the generative model to generate the at least one question sentence; a presentation process for presenting, to a target person, the at least one question sentence that has been generated by the generative model; and a recording process for recording an answer of the target person to the at least one question sentence.

### REFERENCE SIGNS LIST

1 Information processing apparatus
101 Query generating section (query generating means)
102 Generation control section (generation control means)
103 Presentation section (presentation means)
104 Recording section (recording means)
1A Information processing apparatus
101A Query generating section (query generating means)
102A Generation control section (generation control means)
103A Presentation section (presentation means)
104A Recording section (recording means)
106A Inconsistency detecting section (inconsistency detecting means)
107A Survey report generating section (survey report generating means)
2 Generative model

1. An information processing apparatus comprising at least one processor, the at least one processor carrying out:
a query generating process for generating a query that instructs generation of at least one question sentence which asks about a matter serving as a basis for determining a degree of nursing care required;
a generation control process for inputting, into a generative model that has been trained by machine learning to generate a question sentence in accordance with an input query, the query which has been generated in the query generating process, and causing the generative model to generate the at least one question sentence;
a presentation process for presenting, to a target person, the at least one question sentence that has been generated by the generative model; and
a recording process for recording an answer of the target person to the at least one question sentence.

2. The information processing apparatus according to claim 1, wherein the generative model is a language model that has been trained, by machine learning, to learn one or both of (i) a question asked by a survey staff member who carries out a survey for assessment of a nursing care level and (ii) a question asked by an assessment committee member of a care needs assessment committee.

3. The information processing apparatus according to claim 1, wherein the at least one processor carries out a process for inputting, into the generative model, the answer of the target person to the at least one question sentence that has been presented, and causing the generative model to generate a new question sentence related to the at least one question sentence.

4. The information processing apparatus according to claim 1, wherein the at least one processor carries out a survey report generating process for using the recorded answer of the target person to generate a survey report showing a result of an oral survey for assessment of a nursing care level.

5. The information processing apparatus according to claim 1, wherein, in the query generating process, the at least one processor generates the query that includes a document describing a survey item for determining a nursing care level and that instructs generation of a question on the basis of the document.

6. The information processing apparatus according to claim 1, wherein the at least one processor decentralizes or divides, in a period up to a time point at which an oral survey is to be ended, a presentation timing at which a plurality of question sentences that the at least one question sentence comprises are presented.

7. The information processing apparatus according to claim 1, wherein the at least one processor carries out an inconsistency detecting process for detecting inconsistency in the answer of the target person to the at least one question sentence that has been presented.

8. The information processing apparatus according to claim 1, wherein the at least one processor carries out a process for causing a language model that has been trained by machine learning to generate an answer to a question to generate an answer which is to a question from a survey staff member who carries out a survey for assessment of a nursing care level or from an assessment committee member of a care needs assessment committee and which is based on the recorded answer.

9. A care needs assessment support method comprising:
a query generating process for generating a query that instructs generation of at least one question sentence which asks about a matter serving as a basis for determining a degree of nursing care required;
a generation control process for inputting, into a generative model that has been trained by machine learning to generate a question sentence in accordance with an input query, the query which has been generated in the query generating process, and causing the generative model to generate the at least one question sentence;
a presentation process for presenting, to a target person, the at least one question sentence that has been generated by the generative model; and
a recording process for recording an answer of the target person to the at least one question sentence, the query generating process, the generation control process, the presentation process, and the recording process each being carried out by at least one processor.

10. A non-transitory computer-readable recording medium recording therein a care needs assessment support program for causing a computer to function as:
a query generating means for generating a query that instructs generation of at least one question sentence which asks about a matter serving as a basis for determining a degree of nursing care required;
a generation control means for inputting, into a generative model that has been trained by machine learning to generate a question sentence in accordance with an input query, the query which is generated by the query generating means, and causing the generative model to generate the at least one question sentence;
a presentation means for presenting, to a target person, the at least one question sentence that has been generated by the generative model; and
a recording means for recording an answer of the target person to the at least one question sentence.
